(19) United States
(12) Patent Application Publication
(10) Pub. No.: US 2015/0126905 A1
(43) Pub. Date: May 7, 2015

(54) SAFETY SLEEVE SYRINGE

(71) Applicant: Suzanne Lee Lowry, Atlanta, GA (US)

(72) Inventor: Suzanne Lee Lowry, Atlanta, GA (US)

(21) Appl. No.: 14/074,567

(22) Filed: Nov. 7, 2013

(51) Int. Cl. A61B 5/15; A61M 5/32

(52) U.S. Cl. CPC A61B 5/150656; A61M 5/3202
USPC 600/576; 604/197

(57) ABSTRACT

A safety sleeve system for syringes and other needle-based devices prevents accidental needle sticks after use of the syringe. The safety sleeve system allows an easy mechanical cover that, once actuated, locks over the exposed needle for safe coverage and disposal, or, in a sterile situation, allows for exposure of a clean needle. The safety sleeve system minimally alters an existing syringe and allows for identical use and function, with only an outer sleeve cover that follows tracks embossed on the exterior of the existing syringe. This sleeve cover can be twisted and locked in an operation position, where the needle extends from the sleeve cover, or in a safety position, where the needle is encased by the sleeve cover.

### BACKGROUND OF THE INVENTION

The present invention relates to safety devices and, more particularly, to a manual safety sleeve for a syringe or a vacuum blood collection device, such as a Vacutainer® device.

In the healthcare industry, as well as in other industries that use syringes, accidental needle sticks can occur, especially in the handling and disposal of used syringes. Many institutions have strict policies forbidding the recapping of syringes and instructing needle users how to properly and safely dispose of syringes and needles.

Currently, the devices that are commonly used have no safety feature that, once used, prevents accidental sticks or injury from the contaminated needle and further allows the device to be disposed in regular containers without risk to trash handlers.

Some conventional needle covers can bend the needle when actuated and require an awkward process that can cause the operator to be accidently impaled while using the device. Once the needle is bent in storage, the needle can leave the cover, exposing the needle to potentially cause a future needle stick or injury.

As can be seen, there is a need for a safety device for a syringe or syringe-type needle.

### SUMMARY OF THE INVENTION

In one aspect of the present invention, a safety sleeve syringe system comprises a syringe body; a needle extending from an end of the syringe body; at least one raised helical spiral disposed along at least a portion of the syringe body; a safety sleeve fitting over the syringe body; and at least one helical spiral groove disposed inside the safety sleeve, the at least one helical spiral groove mating with the at least one raised helical spiral, wherein twisting the syringe body relative to the safety sleeve moves the safety sleeve along the syringe body, causing the system to move between a safety position, where the needle is encased within the safety sleeve, and an operation position, where the needle extends from the safety sleeve.

In another aspect of the present invention, a safety sleeve syringe system comprises a syringe body; a needle extending from an end of the syringe body; a raised helical spiral disposed along at least a portion of the syringe body; a safety sleeve fitting over the syringe body; a helical spiral groove disposed inside the safety sleeve, the helical spiral groove mating with the raised helical spiral, wherein twisting the syringe body relative to the safety sleeve moves the safety sleeve along the syringe body, causing the system to move between a safety position, where the needle is encased within the safety sleeve, and an operation position, where the needle extends from the safety sleeve; at least one safety lock protrusion on a first end of the syringe body, proximate to the needle, the safety lock protrusion fixing the safety sleeve to the syringe body in the safety position; and at least one operation lock protrusion on a second, opposite end of the syringe body, distal to the needle, the operation lock protrusion fixing the safety sleeve to the syringe body in the operation position.

These and other features, aspects and advantages of the present invention will become better understood with reference to the following drawings, description and claims.

### BRIEF DESCRIPTION OF THE DRAWINGS

FIG. 1 is a perspective view of a safety sleeve syringe, in a safety position with its needle protected, according to an exemplary embodiment of the present invention;

FIG. 2 is a perspective view of the safety sleeve syringe of FIG. 1, in an operation position, with its needle exposed for use;

FIG. 3 is an exploded perspective view of the safety sleeve syringe of FIG. 1;

FIG. 4 is a needle end view of the safety sleeve syringe of FIG. 1;

FIG. 5 is a plunger end view of the safety sleeve syringe of FIG. 1;

FIG. 6 is a cross-sectional view taken along line 6-6 of FIG. 1;

FIG. 7 is a detailed cross-sectional view taken along section 7-7 of FIG. 6;

FIG. 8 is a side view of the safety sleeve syringe of FIG. 1, in a safety position;

FIG. 9 is a side view of the safety sleeve syringe of FIG. 2, in an operation position;

FIG. 10 is a perspective view of a safety sleeve vacuum blood drawing device, in an operation position, according to an exemplary embodiment of the present invention;

FIG. 11 is a side view of the safety sleeve vacuum blood drawing device of FIG. 10 in an operation position; and

FIG. 12 is a side view of the safety sleeve vacuum blood drawing device of FIG. 10, in a safety position.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

Broadly, an embodiment of the present invention provides a safety sleeve system for syringes and other needle-based devices that prevents accidental needle sticks after use of the syringe. The safety sleeve system allows an easy mechanical cover that, once actuated, locks over the exposed needle for safe coverage and disposal, or, in a sterile situation, allows for exposure of a clean needle. The safety sleeve system minimally alters an existing syringe and allows for identical use and function, with only an outer sleeve cover that follows tracks embossed on the exterior of the existing syringe. This sleeve cover can be twisted and locked in an operation position, where the needle extends from the sleeve cover, or in a safety position, where the needle is encased by the sleeve cover.

Once a needle is used, the sleeve cover can be twisted and locked in the safety position. The needle is then covered and the entire device can be disposed. There is no risk of sticks to the user or trash handler. The safety sleeve system of the present invention leaves the operator's hand at the base of the syringe and remote from the needle. After the entire device is disposed of, the system can be easily separated and recycled, as the cover can be made of a plastic that can be melted away and the metallic needle can be recovered by, for example, a magnet.

As used herein, the term syringe is used to generically encompass various needle-containing devices. The syringe can be a typical syringe, with a chamber and plunger for drawing and/or delivering fluids, for example. In some embodiments, other needle-containing devices may be considered as being encompassed by the term syringe. For example, a vacuum blood drawing device, such as a Vacutainer® brand system, can be considered a syringe within the scope of the present invention.

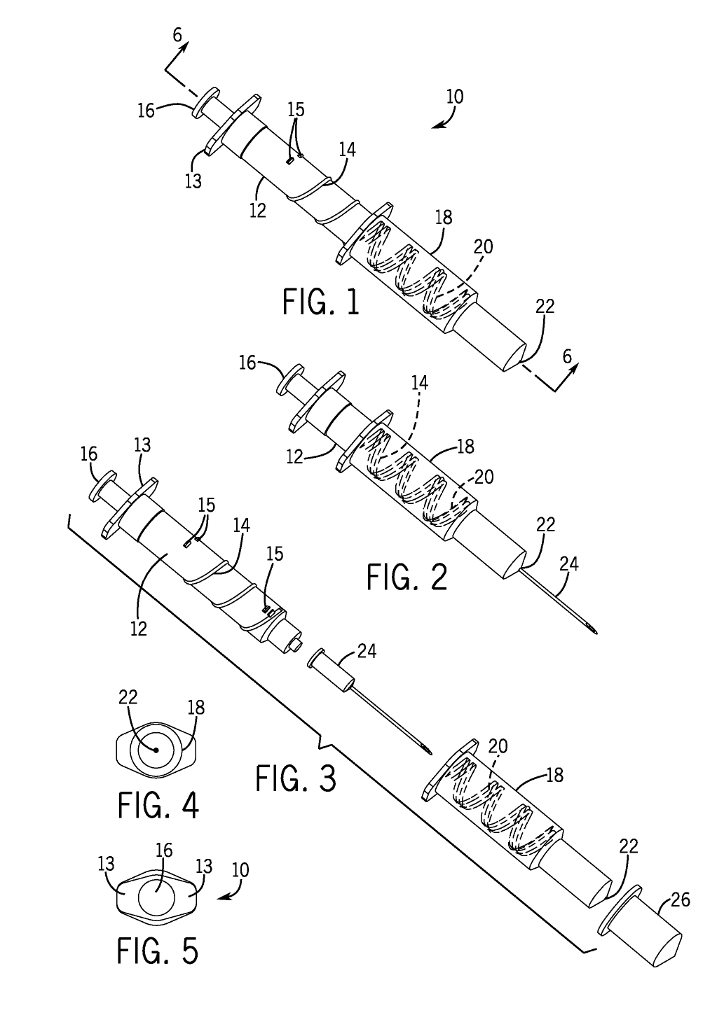

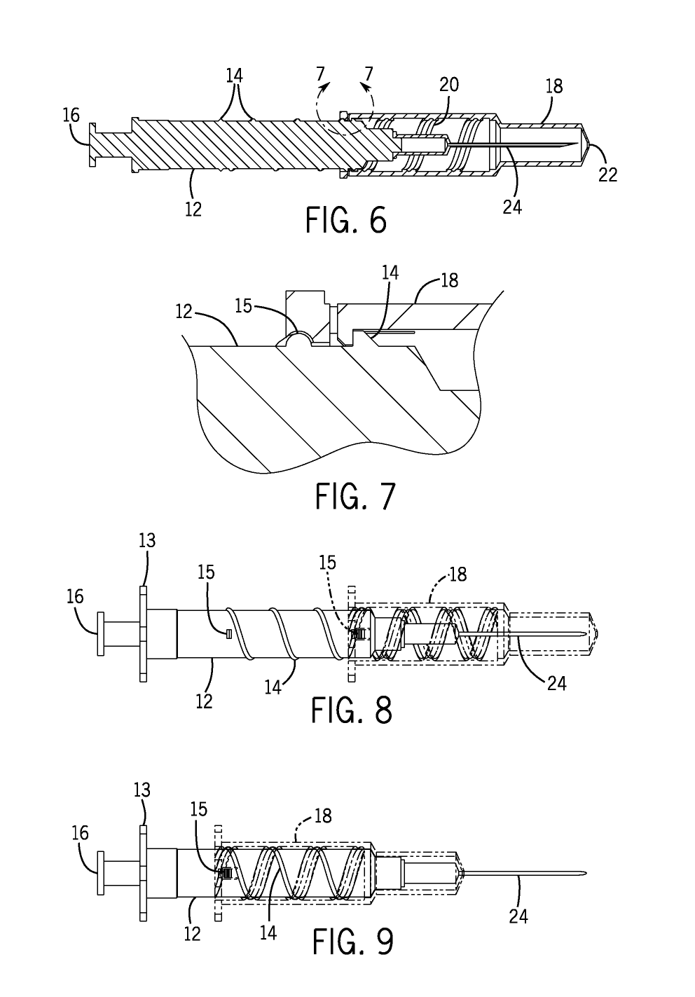

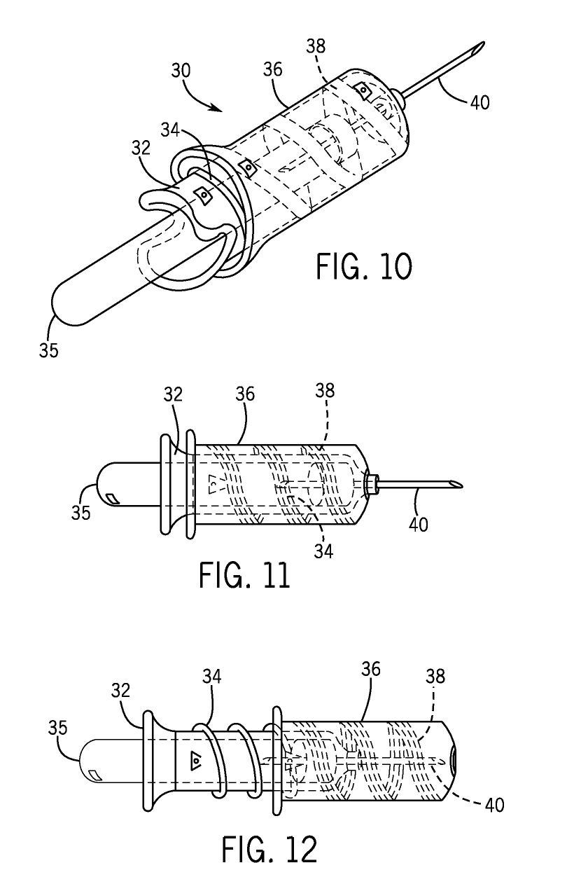

Referring now to FIGS. 1 through 9, a safety sleeve syringe system 10 can include a syringe body 12 having a handle 13 at a first end thereof. A needle 24 can attach to a second, opposite end of the syringe body 12. A plunger 16 can be designed to fit inside the syringe body 12, similar to a conventional syringe.

The exterior of the syringe body 12 can include a raised helical spiral 14, spiraling from the second, opposite end of the syringe body 12 toward the first end thereof. A safety sleeve 18 can be sized to fit over the syringe body 12. The safety sleeve 18 can include a helical spiral groove 20 formed on at least a portion of an inside region thereof.

The helical spiral groove 20 on the safety sleeve 18 can mate with the raised helical spiral 14 on the syringe body 12 to allow the safety sleeve 18 to twist over the syringe body 12 and move between a safety position (as shown in FIGS. 1, 6 and 8, for example) and an operation position (as shown in FIGS. 2 and 9, for example).

A hole 22 can be disposed at the end of the safety sleeve 18 to allow the needle 24 to pass through when the system 10 is changed between its safety position and its operation position. In some embodiments, a cover 26 can be provided over the end of the safety sleeve 18 to cover the hole 22, as may be needed. In some embodiments, the hole 22 can be formed as a penetrable member, such as a rubber septa, through which the needle can extend and be retracted. In this embodiment, the cover 26 may not be necessary.

The safety sleeve syringe system 10 can include a mechanism to cause the syringe body 12 to be held in place, with respect to the safety sleeve 18, in the safety position and in the locked position. In some embodiments, this mechanism can include a safety lock protrusion 15 disposed on the syringe body 12 that can fit into a detent in the safety sleeve 18. The safety lock protrusion 15 can be disposed at opposite ends of the syringe body 12 to permit the system 10 to “lock” in the safety position (FIGS. 6, 7 and 8) and in the operation position (FIG. 9, where the safety lock protrusion 15 may be referred to as an operation lock protrusion 15).

While the Figures show about three spirals for each of the raised helical spiral 14 and the helical spiral groove 20, the number of spirals and their pitch can vary within the scope of the present invention. In addition, while the Figures show, for example, a single raised helical spiral 14, this spiral could be designed in various shapes and configurations, provided that the helical spiral groove 20 of the safety sleeve 18 can mate therewith to allow the safety sleeve 18 to twist to move between the safety position and the operation position.

Referring now to FIGS. 10 through 12, in an alternate embodiment of the present invention, a safety sleeve system 30 can be used on a vacuum blood drawing device, such as a Vacutainer® brand phlebotomy device, in a manner similar to that described above with respect to FIGS. 1 through 9.

A syringe member 32 can have a needle 40 disposed on the end thereof. A raised helical spiral 34 can be disposed about the syringe member 32. A safety sleeve 36 can have a helical spiral groove 38 formed therewithin. The helical spiral groove 38 can mate with the raised helical spiral 34 to allow the safety sleeve 36 to move over the syringe member 32. The safety sleeve 36 can be moved between a safety position, as shown in FIG. 12, and an operation position, as shown in FIGS. 10 and 11. A vial 35 can be inserted into the syringe member 32 for drawing blood through the needle 40 and into the vial 35. Protrusions can be disposed on the syringe member 32 to lock the syringe member 32 in either the safety position or the operation position. The protrusion can be similar to that described above with respect to FIGS. 1 through 9.

The system of the present invention can be used not only with the specific devices shown and described herein, but could be used for any device that has an exposed end with potential for impaling. The safety sleeve can be sized for various sizes of syringes, vacuum blood drawing devices, or the like.

It should be understood, of course, that the foregoing relates to exemplary embodiments of the invention and that modifications may be made without departing from the spirit and scope of the invention as set forth in the following claims.

What is claimed is:

1. A safety sleeve syringe system comprising:
a syringe body;
a needle extending from an end of the syringe body;
at least one raised helical spiral disposed along at least a portion of the syringe body;
a safety sleeve fitting over the syringe body; and
at least one helical spiral groove disposed inside the safety sleeve, the at least one helical spiral groove mating with the at least one raised helical spiral, wherein twisting the syringe body relative to the safety sleeve moves the safety sleeve along the syringe body, causing the system to move between a safety position, where the needle is encased within the safety sleeve, and an operation position, where the needle extends from the safety sleeve.

2. The safety sleeve syringe system of claim 1, further comprising at least one safety lock protrusion on a first end of the syringe body, proximate to the needle, the safety lock protrusion fixing the safety sleeve to the syringe body in the safety position.

3. The safety sleeve syringe system of claim 2, further comprising at least one operation lock protrusion on a second, opposite end of the syringe body, distal to the needle, the operation lock protrusion fixing the safety sleeve to the syringe body in the operation position.

4. The safety sleeve syringe of claim 1, further comprising a plunger fitting into the syringe body.

5. The safety sleeve syringe of claim 1, further comprising a vacuum vial fitting into the syringe body.

6. The safety sleeve syringe of claim 1, further comprising a hole in the safety sleeve configured to permit the needle to pass through when the system is moved between the safety position and the operation position.

7. A safety sleeve syringe system comprising:
a syringe body;
a needle extending from an end of the syringe body;
a raised helical spiral disposed along at least a portion of the syringe body;
a safety sleeve fitting over the syringe body;
a helical spiral groove disposed inside the safety sleeve, the helical spiral groove mating with the raised helical spiral, wherein twisting the syringe body relative to the safety sleeve moves the safety sleeve along the syringe body, causing the system to move between a safety position, where the needle is encased within the safety sleeve, and an operation position, where the needle extends from the safety sleeve;
at least one safety lock protrusion on a first end of the syringe body, proximate to the needle, the safety lock protrusion fixing the safety sleeve to the syringe body in the safety position; and
at least one operation lock protrusion on a second, opposite end of the syringe body, distal to the needle, the operation lock protrusion fixing the safety sleeve to the syringe body in the operation position.

8. The safety sleeve syringe of claim 7, further comprising a plunger fitting into the syringe body.

9. The safety sleeve syringe of claim 7, further comprising a vacuum vial fitting into the syringe body.

10. The safety sleeve syringe of claim 7, further comprising a hole in the safety sleeve configured to permit the needle to pass through when the system is moved between the safety position and the operation position.
